(19) United States
(12) Patent Application Publication
(10) Pub. No.: US 2008/0138242 A1
(43) Pub. Date: Jun. 12, 2008

(54) Method for controlling filter sterilizer for air purifier

(75) Inventors: Young Sik Park, Seoul (KR); Jun-Hyoun Kwon, Seoul (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 12/034,560

(22) Filed: Feb. 20, 2008

Related U.S. Application Data
(62) Division of application No. 11/079,087, filed on Mar. 15, 2005, now abandoned.

(30) Foreign Application Priority Data
Jul. 27, 2004 (KR) 2004-58858

(51) Int. Cl. A61L 2/02

(52) U.S. Cl. 422/22

(57) ABSTRACT

A filter sterilizer for an air purifier and a method for controlling the same. The filter sterilizer includes a filter unit for purifying indoor air, and a controller for controlling the operation of sterilizing the filter. The controller operates both an air blast fan and an ion generator in an air purification mode, to sterilize indoor air, and stops the operation of the air blast fan and operates the ion generator independently in a filter sterilization mode, to sterilize the filter unit. The filter sterilizer sterilizes the filter unit for purifying air without a separate sterilizing apparatus, thereby reducing a user's economic burden and simply sterilizing the filter unit.

### CROSS-REFERENCE TO RELATED APPLICATION

This application claims the benefit of Korean Patent Application No. 2004-58858, filed Jul. 27, 2004, in the Korean Intellectual Property Office, the disclosure of which is incorporated herein by reference.

### BACKGROUND OF THE INVENTION

1. Field of the Invention

An apparatus and method consistent with the present invention relate to a filter sterilizer for an air purifier and a method for controlling the same and, more particularly, to a filter sterilizer for an air purifier, which sterilizes a filter installed in the air purifier, and a method for controlling the filter sterilizer.

2. Description of the Related Art

Air purifiers serve to forcibly circulate indoor air, and cause the inhaled indoor air to pass through a multifunctional filter so that fine particles are filtered out of the indoor air, and supply the obtained clean air to an indoor room.

An air purifier having an electric ion generator supplies cations and anions, together with air discharged to an indoor room by air blast, to the indoor room, thereby sterilizing bacteria in indoor air and removing offensive odors.

When the air purifier is operated for a long period of time, dust and impurities are collected in an air purifying filter, thereby causing propagation of mold and bacteria in the air purifying filter. Accordingly, the contaminated air purifying filter of the air purifier needs to be cleaned or sterilized.

When the filters of the conventional air purifiers are separated from main bodies, and cleaned or washed by users, bacteria attached to the filters are discharged to the air, thereby injuring worker's health and contaminating indoor air.

Further, the conventional air purifiers respectively employ the high-priced air purifying filters having an antibiotic function or additional sterilizing apparatuses attached to the filters, thereby having high production costs and causing economic burden to customers.

### SUMMARY OF THE INVENTION

Illustrative, non-limiting embodiments of the present invention overcome the above disadvantages and other disadvantages not described above. Also, the present invention is not required to overcome the disadvantages described above, and an illustrative, non-limiting embodiment of the present invention may not overcome any of the problems described above.

Therefore, an aspect of the invention is to provide a filter sterilizer for an air purifier, which uses an ion generator, installed to sterilize indoor air, for sterilizing an air purifying filter installed in the air purifier, thereby reducing production costs and simply sterilizing the air purifying filter.

In accordance with one aspect, an apparatus consistent with the present invention provides a filter sterilizer for an air purifier, comprising: a main body provided with inlets and outlets; a filter unit which purifies indoor air drawn through the inlets; an air blast fan which forcibly circulates the indoor air; an ion generator installed in the main body which generates ions; and a controller which stops the operation of the air blast fan and independently operates the ion generator, in a filter sterilization mode, to control an operation for sterilizing the filter unit.

The filter sterilizer may further comprise a switching member for opening and closing a discharge channel for guiding the air purified by the filter unit to the outlets, and the switching member may be controlled so that the discharge channel is closed in the filter sterilization mode.

The switching member may be a damper installed in the discharge channel.

The switching member may be a discharge blade installed in the outlets.

The ion generator may be installed in the discharge channel.

The controller may receive time data from a timer for measuring a sterilizing time of the filter unit, and then sterilize the filter unit for a first predetermined time based on the time data supplied from the timer.

The controller may stop the operation of the ion generator for a second predetermined time based on the time data supplied from the timer so that ozone generated from the ion generator for sterilizing the filter unit is exterminated.

In accordance with another aspect, the present invention provides a filter sterilizer for an air purifier, comprising: an air purifying filter for purifying indoor air; a discharge channel for discharging the air purified by the air purifying filter; a switching member for opening and closing the discharge channel; an ion generator installed in the discharge channel for generating ions; and a controller for closing the discharge channel and operating the ion generator, in a filter sterilization mode, to sterilize the air purifying filter.

In accordance with yet another aspect, the present invention provides a method for controlling a filter sterilizer for an air purifier, which sterilizes a filter unit for purifying indoor air, comprising: determining whether or not a filter sterilization mode is set; closing a discharge channel when it is determined that the filter sterilization mode is set; generating ozone by operating an ion generator installed in the discharge channel; and sterilizing the filter unit using the generated ozone.

An air blast fan installed in the discharge channel may be stopped when it is determined that the filter sterilization mode is set.

The filter sterilization mode may be set based on a measured cumulative operating time of the air purifier.

The filter sterilization mode may be determined by a user through an input unit.

The method may further comprise closing the discharge channel for a designated time and stopping the operation of the ion generator so that the ozone is exterminated after the sterilization of the filter unit.

### BRIEF DESCRIPTION OF THE DRAWINGS

These and/or other aspects and advantages of the invention will become apparent and more readily appreciated from the following description of the exemplary embodiments, taken in conjunction with the accompanying drawings, in which:

FIG. 1 is a perspective view of an air purifier in accordance with one exemplary embodiment the present invention;

FIG. 2 is a cross-sectional view taken along the line I-I of FIG. 1;

FIG. 3 is a cross-sectional view taken along the line II-II of FIG. 1;

FIG. 4 is a schematic view illustrating the air purifier, a discharge channel of which is closed, consistent with the present invention;

FIG. 5 is a block diagram of a filter sterilizer for the air purifier consistent with the present invention;

FIG. 6 is a flow chart illustrating a method for controlling the filter sterilizer for the air purifier consistent with the present invention; and

FIG. 7 is a cross-sectional view of an air purifier consistent with another embodiment of the present invention.

### DETAILED DESCRIPTION OF THE ILLUSTRATIVE, NON-LIMITING EMBODIMENTS OF THE INVENTION

Reference will now be made in detail to the illustrative, non-limiting embodiments of the present invention, an example of which is illustrated in the accompanying drawings, wherein like reference numerals refer to like elements throughout. The illustrative, non-limiting embodiments are described below to explain the present invention by referring to the figures.

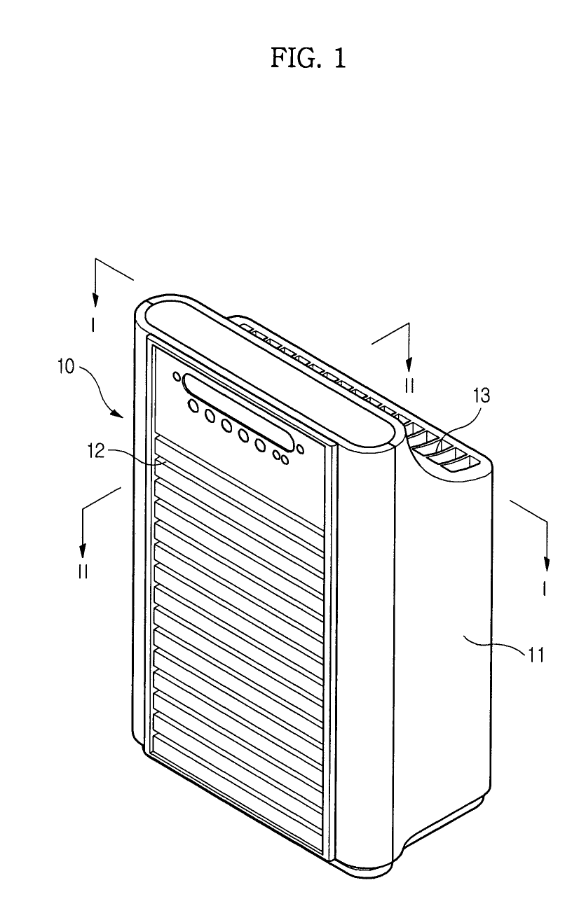

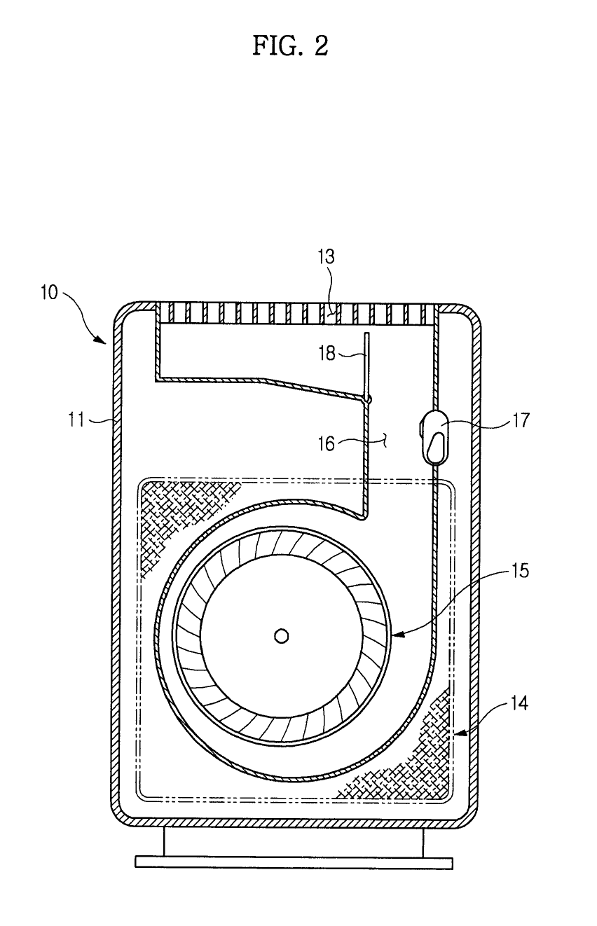

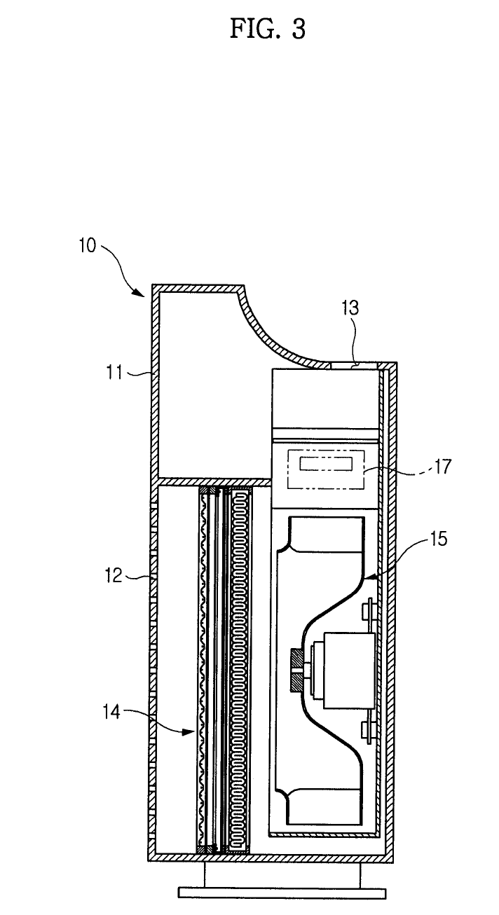

FIG. 1 is a perspective view of an air purifier in accordance with one exemplary embodiment the present invention. FIG. 2 is a cross-sectional view taken along the line I-I of FIG. 1, and FIG. 3 is a cross-sectional view taken along the line II-II of FIG. 1.

As shown in FIGS. 1 to 3, the air purifier 10 in accordance with one embodiment of the present invention comprises a main body 11, a plurality of inlets 12 formed through the front surface of the main body 11, and a plurality of outlets 13 formed through the upper surface of the main body 11.

The main body 11 includes a filter unit 14 for purifying indoor air by filtering out dust or impurities from the indoor air, and an air blast fan 15 for inhaling the indoor air through the inlets 12 and then for forcibly blowing air purified by the filter unit 14 to the outlets 13.

The filter unit 14 is placed between the inlets 12 and the air blast fan 15. The filter unit 14 is a multi-functional filter including a pre-filter for filtering out large particles of dust, an electric dust-collecting filter for collecting charged particles, and a hepa filter for filtering out fine particles of dust.

The main body 11 includes an ion generator 17 and a switching member 18 therein.

The ion generator 17 is an electric ionizer, which includes a discharge electrode, an induction electrode and a needle-shape electrode, and generates cations and anions by applying high voltage to the above electrodes. Kinds and amount of ions generated from the ion generator 17 are adjusted by the applied voltage, and the ion generator 17 is placed in a discharge channel 16.

The switching member 18 serves to open or close the discharge channel 16, and may comprise a damper.

When the air purifier 10 is operated in an air purification mode, both the air blast fan 15 and the ion generator 17 are operated. Indoor air is inhaled into the air purifier 10 through the inlets 12 by the operation of the air blast fan 15, and is purified through the filter unit 14. The purified air passes through the discharge channel 16, and is discharged to an indoor space through the outlets 13, and cations and anions generated from the ion generator 17 are contained by the purified air and discharged to the indoor space, thereby sterilizing bacteria in the indoor air and removing offensive odors.

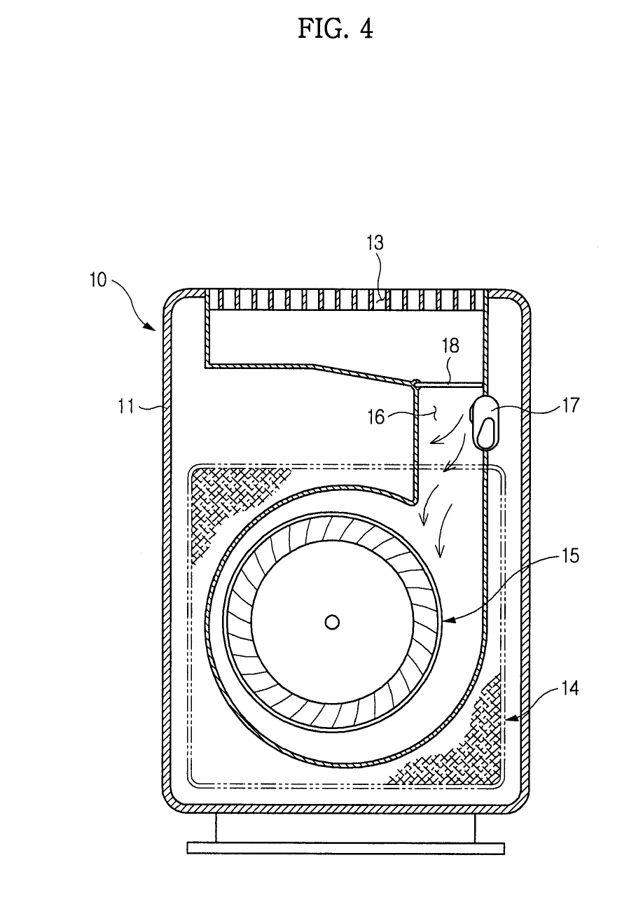

In the case where the air purifier 10 is operated in a filter sterilization mode, the air blast fan 15 is stopped and the ion generator 17 is independently operated. The air blast fan 15 does not generate air blast force, thus not forcibly circulating the indoor air. A large portion of ozone generated from the ion generator 17 descends downwardly, and only a small portion of the ozone moves to the outlets 13. Preferably, but not necessarily, the discharge channel 16 is closed so that the ozone generated from the ion generator 17 is not leaked to the outside of the main body 11 but is used to sterilize the filter unit 14 as shown in FIG. 4. After a designated time elapses, the ozone spreads throughout the filter unit 14, thus being collected by the filter unit 14 or removing bacteria remaining in the filter unit 14.

The ion generator 17 and the switching member 18 are operated by the control of a controller, which will be described later.

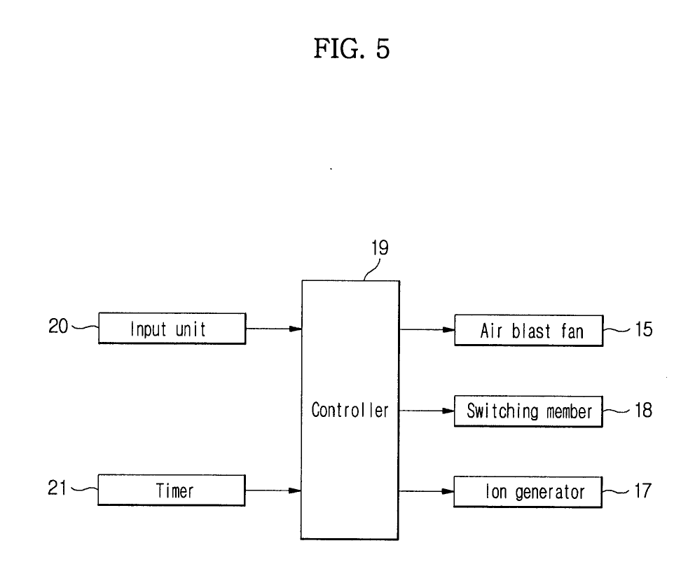

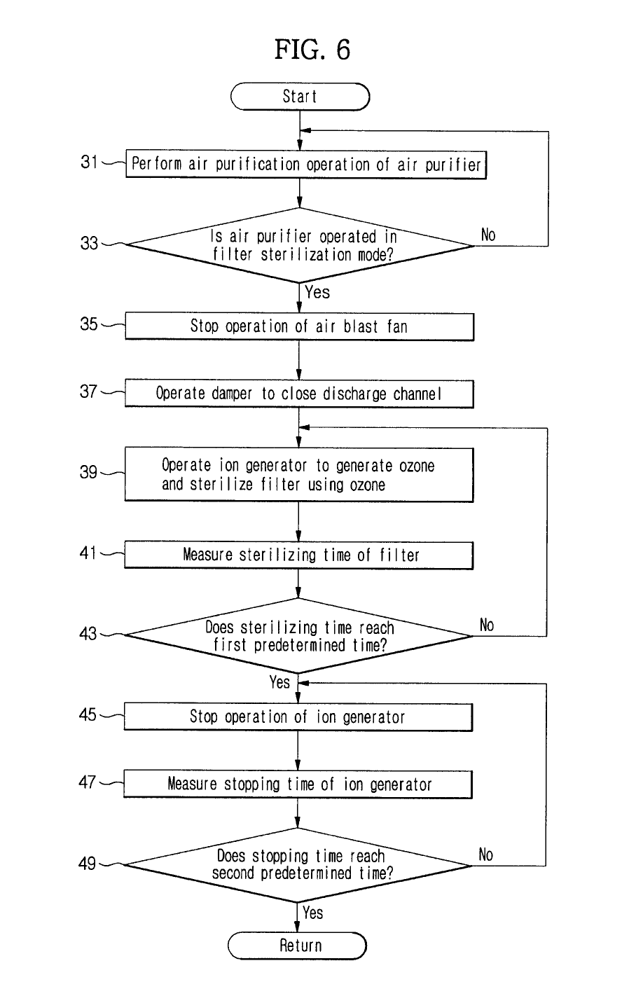

FIG. 5 is a block diagram of a filter sterilizer for the air purifier consistent with the present invention, and FIG. 6 is a flow chart illustrating a method for controlling the filter sterilizer for the air purifier consistent with the present invention.

As shown in FIGS. 5 and 6, the filter sterilizer for the air purifier consistent with the present invention comprises a controller 19 for controlling the overall operations of the air purifier including the air purification operation and the filter sterilization operation. A user sets a desired operation mode, for example, the air purification mode or the filter sterilization mode, through an input unit 20.

A timer 21 measures the operating time of the air purifier and the sterilizing time of the filter unit, and supplies the measured time data to the controller 19. Then, the controller 19 responds to the operation mode set through the input unit 20, and controls the air blast fan 15, the switching member 18 and the ion generator 17.

As shown in FIG. 6, when power is supplied to the air purifier, the controller 19 operates both the air blast fan 15 and the ion generator 17 so that the air purifier is operated in the air purification mode. In this case, the switching member 18 opens the discharge channel 16. Dust and impurities in the indoor air are filtered out by the filter unit 14, and the purified air together with the cations and anions generated from the ion generator 17 is discharged to the indoor space through the outlets 13, thereby removing bacteria floating in the indoor space and offensive odors (S31).

When the air purifier is operated in the air purification mode, the controller 19 determines whether or not the filter sterilization mode is set (S33). Here, the filter sterilization mode is set by at least one selected from two methods, i.e., a manual method, in which the user sets the filter sterilization mode through the input unit 20, and an automatic method, in which the cumulative operating time in every air purification mode is measured by the timer 21 and the measured cumulative operating time is compared to a predetermined time for sterilizing the filter unit 14.

In a case where it is determined that the filter sterilization mode is set as a result of step S33, the controller 19 stops the operation of the air blast fan 15 to stop the forcible circulation of the indoor air (S35), and operates the switching member 18, i.e., the damper, to close the discharge channel 16 (S37). Thereafter, the controller 19 operates the ion generator 17 to generate ozone. The ozone, generated from the ion generator 17, in the air descends downwardly, thereby removing bacteria remaining in the filter unit 14 (S39).

The timer 21 measures the sterilizing time of the filter unit 14, and then supplies the measured sterilizing time to the controller 19 (S41). The controller 19 determines whether or not the sterilizing time of the filter unit 14 measured by the timer 21 reaches a first predetermined time (S43). As a result, in case that it is determined that the sterilizing time of the filter unit 14 measured by the timer 21 does not reach the first predetermined time, the controller 19 controls the method to be returned to the step S39, and in case that it is determined that the sterilizing time of the filter unit 14 measured by the timer 21 reaches the first predetermined time, the controller 19 stops the operation of the ion generator 17 (S45). Here, the timer 21 measures the stopping time of the ion generator 17, and supplies the measured stopping time of the ion generator 17 to the controller 19 (S47). The controller 19 determines whether or not the stopping time of the ion generator 17 reaches a second predetermined time (S49). Here, the second predetermined time is set to allow the ozone to be naturally decomposed in the air inside the main body, thereby preventing the ozone, filling the main body, from being discharged to the outside of the main body, and protecting occupants from the ozone.

In case that it is determined that the stopping time of the ion generator 17 does not reach the second predetermined time as a result of step S49, the controller 19 controls the method to be returned to the step S45 so that the ozone is naturally decomposed, and in case that it is determined that the stopping time of the ion generator 17 reaches the second predetermined time as the result of step S49, the controller 19 recognizes that the ozone is naturally decomposed, and controls the method to be returned to the initial step.

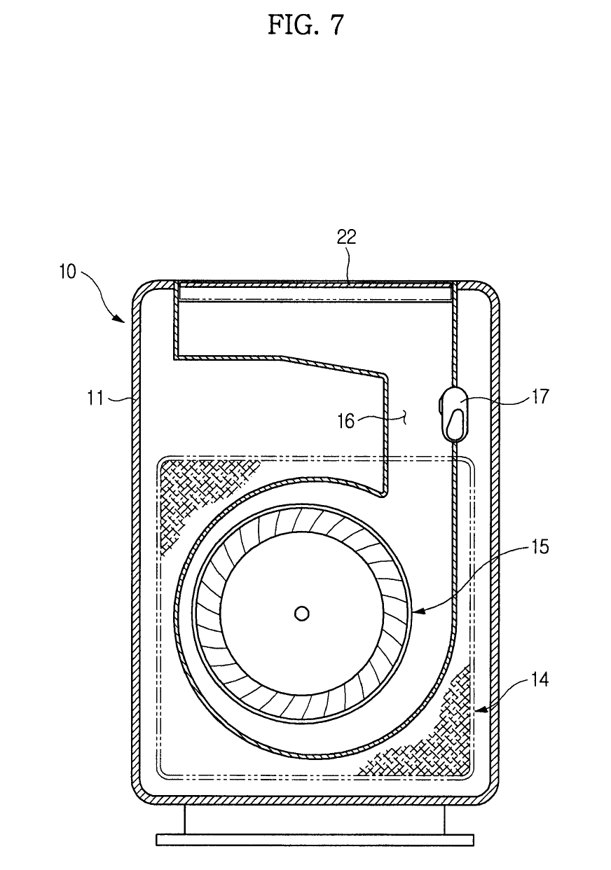

In the above-described embodiment of the present invention, a damper is used as the switching member 18. FIG. 7 is a cross-sectional view of an air purifier consistent with another exemplary embodiment of the present invention. In the further embodiment of the present invention as shown in FIG. 7, a discharge blade 22, which can be opened and closed, is installed in the outlets. Here, in case that the air purifier is operated in the normal air purification mode, the outlets are opened by the discharge blade 22 so that the purified air is discharged to the indoor space. On the other hand, in case that the air purifier is operated in the filter sterilization mode, the outlets are closed by the discharge blade 22 so that the filter unit 14 is sterilized by ozone generated from the ion generator 17.

As apparent from the above description, the present invention provides a filter sterilizer for an air purifier, which uses an ion generator, serving to sterilize indoor air, for sterilizing an air purifying filter without a separate sterilizing apparatus, thereby eliminating a user's economic burden. Further, the filter sterilizer of the present invention simply sterilizes the filter without replacing the filter with a new one, thereby improving the user's convenience.

Although exemplary embodiments of the invention have been shown and described, it would be appreciated by those skilled in the art that changes may be made in these embodiments without departing from the principles and spirit of the invention, the scope of which is defined in the claims and their equivalents.

1. A method of controlling a filter sterilizer for an air purifier, the method comprising:
purifying, by a filter unit indoor air drawn through inlets of a main body of the filter sterilizer;
forcibly circulating, by an air blast fan, the indoor air;
generating ions, by an ion generator installed in the main body of the filter sterilizer;
stopping, by a controller, the operation of the air blast fan and independently operating the ion generator, in a filter sterilization mode, for controlling an operation for sterilizing the filter unit;
receiving by the controller time data from a timer for measuring a sterilizing time of the filter unit, and then sterilizing the filter unit for a first predetermined time based on the time data supplied from the timer; and
stopping by the controller the operation of the ion generator for a second predetermined time based on the time data supplied from the timer so that ozone generated from the ion generator for sterilizing the filter unit is exterminated.

2. The method according to claim 1, further comprising opening and closing a discharge channel, by a switching member, for guiding the air purified by the filter unit to outlets of the main body of the filter sterilizer, and
controlling the switching member so that the discharge channel is closed, in the filter sterilization mode.

3. The method according to claim 2, wherein the switching member comprises a damper installed in the discharge channel.

4. The method according to claim 2, wherein the switching member comprises a discharge blade installed in the outlets.

5. The method according to claim 2, further comprising installing the ion generator in the discharge channel.

6.-8. (canceled)

9. A method for controlling a filter sterilizer for an air purifier, which sterilizes a filter unit for purifying indoor air, the method comprising:
determining whether or not a filter sterilization mode is set;
closing a discharge channel when it is determined that the filter sterilization mode is set;
generating ozone by operating an ion generator installed in the discharge channel; and
sterilizing the filter unit using the generated ozone.

10. The method according to claim 9,
wherein an air blast fan installed in the discharge channel is stopped when it is determined that the filter sterilization mode is set.

11. The method according to claim 9,
wherein the filter sterilization mode is set based on a measured cumulative operating time of the air purifier.

12. The method according to claim 9,
wherein the filter sterilization mode is determined by a user through an input unit.

13. The method according to claim 9, further comprising closing the discharge channel for a designated time and stopping the operation of the ion generator so that the ozone is exterminated after the sterilization of the filter unit.

14. The method according to claim 1, further comprising setting the filter sterilization mode based on a measured cumulative operating time of the air purifier.

15. The method according to claim 1, further comprising generating, by said ion generator, cations and anions.

16. The method according to claim 1, wherein said ion generator is an electronic ionizer comprising a discharge electrode, an induction electrode and a needle-shape electrode.

17. The method according to claim 1, wherein said filter unit is a multi-functional filter comprising a pre-filter which filters out large particles of dust, an electric dust-collecting filter which collects charged particles, and a hepa filter which filters out fine particles of dust.

18. The method according to claim 1, wherein said filter unit comprises a plurality of inlets.

19. The method according to claim 1, further comprising housing said controller within the filter sterilizer.

20. The method according to claim 1, further comprising, stopping, by the controller, the operation of the air blast fan and independently operating the ion generator, upon selection of a filter sterilization mode by the controller.

21. A method of controlling a filter sterilizer for an air purifier, the filter sterilizer comprising:
a main body provided with inlets and outlets;
a filter unit which purifies indoor air drawn through the inlets;
an air blast fan which forcibly circulates the indoor air;
an ion generator installed in the main body and which generates ions; and
a controller which stops the operation of the air blast fan and independently operates the ion generator, in a filter sterilization mode, to control an operation for sterilizing the filter unit;
the method comprising:
determining whether or not the filter sterilization mode is set;
closing a discharge channel when it is determined that the filter sterilization mode is set;
generating ozone by operating the ion generator; and
sterilizing the filter unit using the generated ozone.
